(19) United States
(12) Patent Application Publication
(10) Pub. No.: US 2023/0087644 A1
(43) Pub. Date: Mar. 23, 2023

(54) Probe for Specifically Detecting Pathological Collagens, and Preparation Method Therefor and Use Thereof

(71) Applicant: LANZHOU UNIVERSITY, Gansu (CN)

(72) Inventors: Jianxi XIAO, Gansu (CN); Xiangdong CAI, Gansu (CN)

(21) Appl. No.: 17/790,596

(22) PCT Filed: Sep. 24, 2020
(86) PCT No.: PCT/CN2020/117354

§ 371 Date: Jul. 1, 2022

(30) Foreign Application Priority Data
Jan. 2, 2020 (CN) 202010002171.3

(51) Int. Cl. G01N 33/68; G01N 33/533

(52) U.S. Cl. CPC G01N 33/6887; G01N 33/533; G01N 2333/78; G01N 2800/10

(57) ABSTRACT

The invention belongs to the technical field of collagen detection, and in particular relates to a peptide probe for the specific detection of pathological collagen in tissues, the preparation methods and applications. The peptide probe disclosed in this invention comprises a peptide sequence (Gly-Hyp-Pro)ₙ and a signal molecule modified at the N-terminal of the peptide sequence (Gly-Hyp-Pro)ₙ. The peptide sequence (Gly-Hyp-Pro)ₙ can maintain a stable single-stranded conformation without introducing other components, and will not form a trimer state at all. After the N-terminal of the peptide sequence is connected with a signal molecule, it can be used as a peptide probe for the detection of pathological collagen. The preparation method of the peptide probe is simple. Compared with the existing peptide (GPO, GPP or GOO) probe, it can continuously maintain 100% single-stranded structure, and the concentration of the single-chain probe can be accurately quantified. Moreover, this peptide probe can recognize pathological collagen in tissues of diseases such as arthritis, which has wide prospects of application in fields such as early diagnosis and efficacy evaluation of collagen-related diseases.

### CROSS-REFERENCE TO RELATED APPLICATIONS

This application is a national phase application of the international application No. PCT/CN2020/117354 filed on Sep. 24, 2020, which claims the priority of the Chinese patent application No. CN 2020100021713 filed on Jan. 2, 2020, both of which are hereby incorporated by reference.

### TECHNICAL FIELD

The invention belongs to the technical field of collagen detection, and in particular relates to a peptide probe for the specific detection of pathological collagen in tissues, the preparation methods and applications.

### BACKGROUND TECHNOLOGY

Collagen, the most abundant protein in mammals, is a major component of the extracellular matrix. Collagen plays a key role in tissue formation and maintenance of homeostasis, and its abnormal remodeling is thought to be one of the key triggers of diseases such as fibrosis, tumors and arthritis. Excessive deposition of collagen is often found in fibrosis-related diseases, which can disrupt normal tissue structure and impair organ function. The abnormal distribution of different types of collagen is believed to be closely related to tumor growth, invasion and metastasis. In addition, excessive degradation of type II collagen has been widely recognized as a major factor in the induction of rheumatoid arthritis and osteoarthritis. Therefore, as an important biomarker of arthritis, tumor and other diseases, the efficient detection of pathological collagen is crucial for the diagnosis, treatment and prognosis evaluation of these serious diseases.

Due to its special structure, the targeted detection of pathological collagen has always been very difficult. At present, collagen is mainly detected by antibody-antigen immunoassay or traditional dye-based kit. However, these methods have defects such as complex steps, weak affinity and poor specificity. Moreover, these methods are completely unable to distinguish normal collagen from pathological collagen. Recent studies have found that peptide probes with (Gly-Pro-Hyp)n repetitive sequences can specifically bind to some of unfolding sites in pathological collagen while remaining single-stranded. However, such peptide probes spontaneously form trimers and lose their ability to bind to the pathological collagen. Although pretreatment methods such as heating can transform them into a single-stranded state, their strong ability to form trimers makes the quantification of probe monomers very difficult, and there is a risk of tissue damage, which greatly limits their prospects for clinical application.

Many approaches have been attempted to inhibit the trimer formation of peptide probes targeting pathological collagen. One of the effective strategies is to modify the side chains of peptide probes with larger groups, including photocleavable nitrobenzyl groups, fluorescent dyes such as carboxyfluorescein, or larger unnatural amino acids such as (2S,4S)-4-fluoroproline. These modifications to the (Gly-Pro-Hyp)n sequence can drive the peptide probe to form a single-stranded conformation, but they greatly increase the difficulty and cost of peptide probe synthesis, and significantly increase the toxicity risk of peptide probes for in vivo imaging applications.

Natural amino acids have the advantages of high biocompatibility, low toxicity, convenient source and low cost. Therefore, the construction of peptide probes targeting pathological collagen that can maintain single-stranded conformation by natural amino acids has been attracting much attention. Chinese patent CN110129029A discloses a functional probe of single-stranded collagen peptide induced by charge repulsion, which is obtained by attaching charged amino acids at one end of GPO (Gly-Pro-Hyp), GPP (Gly-Pro-Pro) or GOO (Gly-Hyp-Hyp) repetitive sequences and modifying luminescent substances at the other end. The introduction of multiple charged amino acids increases the length of peptide probe, which further complicates the process of probe synthesis and increases the cost of probe synthesis. Chinese patent CN107530454A discloses a peptide conjugate, wherein the conjugate is a dimer collagen peptide formed by connecting two collagen peptides through a linker. However, the peptide conjugate has a complex structure and is difficult to synthesize. It is easy to mismatch in the preparation process, resulting in large differences in structures of the obtained peptide conjugates, which will greatly affect the detection results.

In view of the problems existing in current technologies, the inventors, after many experiments and studies, found that the peptide containing multiple GOP (Gly-Hyp-Pro) repetitive sequences can maintain a stable single-stranded conformation without introducing other components, and has no ability of trimerization in nature. The peptide sequence can be used as a peptide probe for the targeted detection of pathological collagen after the signal molecule is attached at N-terminal. The peptide probe is easy to prepare and can maintain a 100% single-stranded structure without forming trimer state at all compared to existing peptide (GPO, GPP or GOO) probes, so that the concentration of the single-stranded probe can be accurately quantified, and it does not require pretreatment steps such as heating before use, avoiding the potential risk of tissue damage. Compared with the common Masson Trichrome staining method, this peptide probe shows unique high selectivity, and only binds to pathological collagen without interference from normal collagen. This peptide probe can be used for the targeted identification of pathological collagen in tissues such as arthritis, and can be used for staining of paraffin sections and frozen sections. It has great potential in histopathological staining and other clinical applications, and has a wide application prospect in the early diagnosis and efficacy evaluation of collagen-related diseases.

### CONTENTS OF THE INVENTION

In view of the above technical problems, the purpose of the present invention is to provide a peptide probe for detecting pathological collagen. The peptide probe comprises a peptide sequence (Gly-Hyp-Pro)ₙ and a signal molecule X modified at the N-terminal of the peptide sequence (Gly-Hyp-Pro)ₙ, where n is an integer greater than 6.

Preferably, the signal molecule X is fluorescein dye, coumarin dye, rhodamine dye, cyanine dye, BODIPY dye, tetraphenylethylene dye, and one or more of hexaphenylmethylsilane dyes, stilbene anthracene dyes, semiconductor quantum dots, carbon quantum dots, perovskite quantum dots, rare earth ion complexes, metal frame materials, up-conversion rare earth nanomaterials and long afterglow nanomaterials.

Preferably, the signal molecule X is carboxyl fluorescein FAM.

Preferably, the signal molecule X and the peptide sequence (Gly-Hyp-Pro)n are connected through a linker Ahx, and the peptide probe sequence is FAM-Ahx-(Gly-Hyp-Pro)ₙ.

Preferably, the n is any integer between 8 and 12.

The present invention also aims to provide a method for preparing peptide probes, which includes:

(1) Solid-phase synthesis of peptide resin (Gly-Hyp-Pro)ₙ;

(2) After 4eq of signal molecules, HOBt and HBTU are dissolved in DMF and activated at low temperature for 10-30 min, 4-10 eq of DIEA is added to the solution to obtain a mixture;

(3) Add the mixture prepared in step (2) to the resin described in step (1) and react for 12-48 h away from light;

(4) After treating the resin from step (3) with the cutting fluid for 2-4 h, add ice diethyl ether, and the resulting precipitate is referred to as the peptide probe. Wherein, the cutting fluid consists of trifluoroacetic acid, free radical catching agent and water in a volume ratio of 95:2.5:2.5.

The present invention also aims to provide an application of a peptide probe in the preparation of detection reagents, and/or kits, and/or imaging reagents for detecting the content of pathological collagen.

The present invention also aims to provide a detection reagent containing the above-mentioned peptide probe.

The present invention also aims to provide a detection kit containing the above-mentioned peptide probe.

The present invention also aims to provide a tissue imaging reagent containing the above-mentioned peptide probe.

The beneficial effects of the present invention are as follows: \textcircled{1} The present invention first finds that the peptide containing more than 6 GOP (Gly-Hyp-Pro) repetitive sequences can maintain a stable single-stranded conformation without introducing other components, and has no tendency of trimerization at all; \textcircled{2} After the signal molecule is attached to the N-terminal of this peptide sequence, a peptide probe is prepared for the specific detection of pathological collagen. Compared with the existing peptide (GPO, GPP or GOO) probes, it can maintain a 100% single-stranded structure without forming trimer state at all, so that the concentration of the single-stranded probe can be accurately quantified, and it does not require pretreatment steps such as heating before use, avoiding the potential risk of tissue damage; \textcircled{3} Compared with the common Masson Trichrome staining method, this peptide probe shows unique high selectivity, only binds to pathological collagen without interference from normal collagen, and has good specificity; \textcircled{4} The peptide probe provided by the present invention has good fluorescence properties, can be used as a tissue imaging reagent to target and identify pathological collagen in tissues such as arthritis, and can be used for staining of paraffin sections and frozen sections. It has great potential in histopathological staining and other clinical applications, and has a wide application prospect in the early diagnosis and efficacy evaluation of collagen-related diseases; \textcircled{5} The peptide probe provided by the present invention does not contain unnatural amino acids, has good biocompatibility and is easy to prepare.

### DESRIPTION OF FIGURES

FIG. 1 Fluorescence staining performance of the peptide probes;

FIG. 2 Colorimetric comparison of peptide probe solutions;

FIG. 3 Comparison of fluorescence intensity of peptide probe solutions;

FIG. 4 Comparison of thermal stability curves of peptide probes;

FIG. 5 Fluorescence microscope images of mouse intestinal tissue stained by peptide probes;

FIG. 6 Fluorescence microscope images of mouse ear tissue stained by peptide probe;

FIG. 7 Fluorescence microscope images of pathological section of fibrocartilage in human osteoarthritis stained by peptide probe;

FIG. 8 Fluorescence microscope images of pathological section of hyaline cartilage in human osteoarthritis stained by peptide probe.

### MODE OF CARRYING OUT THE INVENTION

The technical solutions of the present invention are further described below in combination with embodiments, but the protection scope of the present invention is not limited to the following.

The peptide probes GOP-8, GOP-10 and GOP-12 prepared in the following embodiments can specifically bind to pathological collagen, and GOP-10 is taken as an example for tissue fluorescence staining However, the peptide probes described in the present invention are not limited to GOP-10, and other probes also have good fluorescence staining ability and can specifically bind to pathological collagen.

### Embodiment 1 Preparation of Peptide Probe GOP-4

1. Design of peptide probe

The sequence of the peptide probe designed in this embodiment is: FAM-Ahx-(GOP)₄-NH₂, in which FAM is carboxyl fluorescein.

2. Solid-phase synthesis of peptide sequence Ahx-(GOP)₄

(1) Add 100 mg Rink amine resin into the reactor with sieve plate, and use 5 mL dichloromethane to swell the resin;

(2) Remove the N-terminal Fmoc protective group by 20% piperidine/N,N-dimethylformamide (DMF) solution, and observe the chromogenic reaction to detect that the protective group is completely removed;

(3) Dissolve the amino acid (4eq) whose N-terminal is protected by Fmoc, HOBt (4eq) and HBTU (4eq) in DMF, activate the solution at low temperature for 20 min, then dropwise add DIEA (6eq) into the solution, and add the solution to the reactor after mixing for 3 h reaction.

(4) After the reaction, extract the reaction solution from the reactor, and wash the resin by 10 mL DMF for 3 times and 10 mL DCM for 2 times, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GOP)₄ of the target sequence is synthesized.

3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GOP)₄ resin, and react in the dark for 24 h;

(2) Wash the resin three times in turn with DCM and methanol respectively, drain the resin, add the cutting fluid (TFA:TIS:water=95:2.5:2.5), and react for 2.5 h;

(3) Add ice ethyl ether to the reaction solution to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GOP)₄-NH₂. The crude product is purified by RP-HPLC to obtain the peptide probe GOP-4.

### Embodiment 2 Preparation of Peptide Probe GOP-6

1. Design of peptide probe

The sequence of the designed peptide probe is: FAM-Ahx-(GOP)₆-NH₂, in which FAM is carboxyl fluorescein.

2. Solid-phase synthesis of peptide sequence Ahx-(GOP)₆

(1) Add 100 mg Rink amine resin into the reactor with sieve plate, and use 5 mL dichloromethane to swell the resin;

(2) Remove the N-terminal Fmoc protective group by 20% piperidine/DMF solution, and observe the chromogenic reaction to detect that the protective group is completely removed;

(3) Dissolve the amino acid (4eq) whose N-terminal is protected by Fmoc, HOBt (4eq) and HBTU (4eq) in DMF, activate the solution at low temperature for 20 min, then dropwise add DIEA (6eq) into the solution, and add the solution to the reactor after mixing for 3 h reaction.

(4) After the reaction, extract the reaction solution from the reactor, and wash the resin by 10 mL DMF for 3 times and 10 mL DCM for 2 times, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GOP)₆ of the target sequence is synthesized.

3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GOP)₆ resin, and react in the dark for 24 h;

(2) Wash the resin three times in turn with DCM and methanol respectively. Drain the resin, add the cutting fluid (TFA:TIS:water=95:2.5:2.5), and react for 2.5 h;

(3) Add the reaction solution to ice ethyl ether to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GOP)₆-NH₂. The crude product is purified by RP-HPLC to obtain the peptide probe GOP-6.

### Embodiment 3 Preparation of Peptide Probe GOP-8

1. Design of peptide probe

The sequence of the designed peptide probe is: FAM-Ahx-(GOP)₈-NH₂, in which FAM is carboxyl fluorescein.

2. Solid-phase synthesis of peptide sequence Ahx-(GOP)₈

(1) Add 100 mg Rink amine resin into the reactor with sieve plate, and use 5 mL dichloromethane to swell the resin; (2) Remove the N-terminal Fmoc protective group by 20% piperidine/DMF solution, and observe the chromogenic reaction to detect that the protective group is completely removed;

(3) Dissolve the amino acid (4eq) whose N-terminal is protected by Fmoc, HOBt (4eq) and HBTU (4eq) in DMF, activate the solution at low temperature for 20 min, then dropwise add DIEA (6eq) into the solution, and add the solution to the reactor after mixing for 3 h reaction.

(4) After the reaction, extract the reaction solution from the reactor, and wash the resin by 10 mL DMF for 3 times and 10 mL DCM for 2 times, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GOP)₈ of the target sequence is synthesized.

3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GOP)s resin, and react in the dark for 24 h;

(2) Wash the resin three times in turn with DCM and methanol respectively, drain the resin, add the cutting fluid (TFA:TIS:water=95:2.5:2.5), and react for 2.5 h;

(3) Add the reaction solution to ice ethyl ether to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GOP)₈-NH₂. The crude product is purified by RP-HPLC to obtain the peptide probe GOP-8.

### Embodiment 4 Preparation of Peptide Probe GOP-10

1. Design of peptide probe

The sequence of the designed peptide probe is: FAM-Ahx-(GOP)₁₀-NH₂, in which FAM is carboxyl fluorescein.

2. Solid-phase Synthesis of Peptide Sequence Ahx-(GOP)₁₀

(1) Add 100 mg Rink amine resin into the reactor with sieve plate, and use 5 mL dichloromethane to swell the resin;

(2) Remove the N-terminal Fmoc protective group by 20% piperidine/DMF solution, and observe the chromogenic reaction to detect that the protective group is completely removed;

(3) Dissolve the amino acid (4eq) whose N-terminal is protected by Fmoc, HOBt (4eq) and HBTU (4eq) in DMF, activate the solution at low temperature for 20 min, then dropwise add DIEA (6eq) into the solution, and add the solution to the reactor after mixing for 3 h reaction.

(4) After the reaction, the reaction solution is extracted from the reactor, and the resin is washed three times with 10 mL DMF and twice with 10 mL DCM, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GOP)₁₀ of the target sequence is synthesized.

3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GOP)₁₀ resin, and react in the dark for 24 h;

(2) Wash the resin three times in turn with DCM and methanol respectively. Drain the resin, add the cutting fluid (TFA:TIS:water=95:2.5:2.5), and react for 2.5 h;

(3) Add the reaction solution to ice ethyl ether to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GOP)₁₀-NH₂. The crude product is purified by RP-HPLC to obtain the peptide probe GOP-10.

### Embodiment 5 Preparation of Peptide Probe GOP-12

1. Design of peptide probe

The sequence of the designed peptide probe is: FAM-Ahx-(GOP)₁₂-NH₂, in which FAM is carboxyl fluorescein.

2. Solid-phase Synthesis of Peptide Sequence Ahx-(GOP)₁₂

(1) Add 100 mg Rink amine resin into the reactor with sieve plate, and use 5 mL dichloromethane to swell the resin;

(2) Remove the N-terminal Fmoc protective group by 20% piperidine/DMF solution, and observe the chromogenic reaction to detect that the protective group is completely removed;

(3) Dissolve N-terminal amino acid (4eq) protected by Fmoc with HOBt (4eq) and HBTU (4eq) in DMF, activate at low temperature for 20 min, add DIEA (6eq) to the solution, mix the solution and add it to the reactor for reaction of 3 hours.

(4) After the reaction, extract the reaction solution from the reactor, and wash the resin by 10 mL DMF for 3 times and 10 mL DCM for 2 times, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GOP)₁₂ of the target sequence is synthesized.

3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GOP)₁₂ resin, and react in the dark for 24 h;

(2) Wash the resin three times in turn with DCM and methanol respectively. Drain the resin, add the cutting fluid (TFA:TIS:water=95:2.5:2.5), and react for 2.5 h;

(3) Add the reaction solution to ice ethyl ether to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GOP)₁₂-NH₂. The crude product is purified by RP-HPLC to obtain the peptide probe GOP-₁₂.

### CONTRASTING EXAMPLE 1

### Preparation of Peptide Probe GPP-10

1. Design of peptide probe

The sequence of the designed peptide probe is: FAM-Ahx-(GPP)₁₀-NH₂, in which FAM is carboxyl fluorescein.

2. Solid-phase Synthesis of Peptide Sequence Ahx-(GPP)₁₀

(1) Add 100 mg Rink amine resin into the reactor with sieve plate, and use 5 mL dichloromethane to swell the resin;

(2) Remove the N-terminal Fmoc protective group by 20% piperidine/DMF solution, and observe the chromogenic reaction to detect that the protective group is completely removed;

(3) Dissolve the amino acid (4eq) whose N-terminal is protected by Fmoc, HOBt (4eq) and HBTU (4eq) in DMF, activate the solution at low temperature for 20 min, then dropwise add DIEA (6eq) into the solution, and add the solution to the reactor after mixing for 3 h reaction.

(4) After the reaction, the reaction solution is extracted from the reactor, and the resin is washed three times with 10 mL DMF and twice with 10 mL DCM, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GPP)₁₀ of the target sequence is synthesized.

3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GPP)₁₀ resin, and react in the dark for 24 h;

(2) Wash the resin three times in turn with DCM and methanol respectively. Drain the resin, add cutting fluid (TFA:TIS:water=95:2.5:2.5), and react for 2.5 h;

(3) Add the reaction solution to ice ethyl ether to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GPP)₁₀-NH₂. The crude product is purified by RP-HPLC to obtain the peptide probe GPP-10.

### CONTRASTING EXAMPLE 2

### Preparation of Peptide Probe GPO-10

1. Design of peptide probe

The sequence of the designed peptide probe is: FAM-Ahx-(GPO)₁₀-NH₂, in which FAM is carboxyl fluorescein.

2. Solid-phase Synthesis of Peptide Sequence Ahx-(GPO)₁₀

(1) Add 100 mg Rink amine resin into the reactor with sieve plate, and use 5 mL dichloromethane to swell the resin;

(2) Remove the N-terminal Fmoc protective group by 20% piperidine/DMF solution, and observe the chromogenic reaction to detect that the protective group is completely removed;

(3) Dissolve the amino acid (4eq) whose N-terminal is protected by Fmoc, HOBt (4eq) and HBTU (4eq) in DMF, activate the solution at low temperature for 20 min, then dropwise add DIEA (6eq) into the solution, and add the solution to the reactor after mixing for 3 h reaction.

(4) After the reaction, extract the reaction solution from the reactor, and wash the resin by 10 mL DMF for 3 times and 10 mL DCM for 2 times, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GPO)₁₀ of the target sequence is synthesized.

3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GPO)₁₀ resin, and react in the dark for 24 h;

(2) Wash the resin three times in turn with DCM and methanol respectively. Drain the resin, add the cutting fluid (TFA:TIS:water=95:2.5:2.5), and react for 2.5 h;

(3) Add the reaction solution to ice ethyl ether to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GPO)₁₀-NH₂. The crude product is purified by RP-HPLC to obtain the peptide probe GPO-10.

### CONTRASTING EXAMPLE 3

### Preparation of Peptide Probe GOO-10

1. Design of peptide probe

The sequence of the designed peptide probe is: FAM-Ahx-(GOO)₁₀-NH₂, in which FAM is carboxyl fluorescein.

2. Solid-phase Synthesis of Peptide Sequence Ahx-(GOO)₁₀

(1) Add 100 mg Rink amine resin into the reactor with sieve plate, and use 5 mL dichloromethane to swell the resin;

(2) Remove the N-terminal Fmoc protective group by 20% piperidine/DMF solution, and observe the chromogenic reaction to detect that the protective group is completely removed;

(3) Dissolve the amino acid (4eq) whose N-terminal is protected by Fmoc, HOBt (4eq) and HBTU (4eq) in DMF, activate the solution at low temperature for 20 min, then dropwise add DIEA (6eq) into the solution, and add the solution to the reactor after mixing for 3 h reaction.

(4) After the reaction, the reaction solution is extracted from the reactor, and the resin is washed three times with 10 mL DMF and twice with 10 mL DCM, respectively. Observe the chromogenic reaction to detect that the condensation of amino acids is complete, and treat the resin with 20% piperidine/DMF solution three times for 5 min, 5 min and 15 min respectively. Wash the resin by 10 mL DMF for 3 times and 10 mL DCM for 2 times, respectively, and observe the chromogenic reaction to detect that the protective group is completely removed;

(5) Repeat steps (3) and (4) until the peptide Ahx-(GOO)₁₀ of the target sequence is synthesized. 3. Peptide sequences modified by signal molecules

(1) Weigh and take carboxyfluorescein (4eq), HOBt (4eq) and HBTU (4eq), dissolve them in DMF, activate them at low temperature for 20 min, then drop DIEA (6eq) into the solution, add the mixture into the synthesized Ahx-(GOO)₁₀ resin, and react in the dark for 24 h;

(2) Wash the resin three times in turn with DCM and methanol respectively. Drain the resin, add the cutting fluid (TFA:TIS:water=95:2.5:2.5), and react for 2.5 h;

(3) Add the reaction solution to ice ethyl ether to precipitate the peptide. Centrifuge the precipitate, dissolve the precipitate with a small amount of TFA, add excessive ice ethyl ether to precipitate again, centrifuge and collect the precipitate, wash the precipitate with ice ethyl ether twice, and then air dry to obtain crude peptide FAM-Ahx-(GOO)₁₀-NH₂. Purify the crude product by reversed-phase liquid chromatography to obtain peptide probe GOO-10.

### Embodiment 6 Performance Determination of Peptide Probe

### 1. Determination of staining

Take the peptide probe prepared in Embodiment 1-5 for staining experiment. Paraffin staining of sternum: Embed mouse sternum tissue sections by paraffin and bake at 75° C. for 2 h; After cooling, treat the tissue with xylene twice, each time for 10 min; Then treat the slice with ethanol of gradient concentration; Finally, use ultrapure water to wash the slices twice. After removing the paraffin, treat the sternum tissue with ultrapure water at 85° C. for 10 minutes to denature collagen. Add blocking solution to the tissue section, leave it for 30 min, then suck up the solution, stain with 100 μL and 15 μM of the above peptide probe solutions, and incubate at 4° C. for 4 h; After removing the staining solution, wash with 1×PBS for 3 times, and wash the unbound probe with the washing solution; Drop mount solution, add cover slide, observe and take photos with fluorescence microscope.

Staining of frozen tail tissue: Treat tail tissue with 1% sodium dodecyl sulfate for 24 hours to denature collagen, and prepare the frozen sections. Put the slices in cold methanol and fix them at −20° C. for 10 min. Wash the frozen sections twice with PBS, add blocking solution to the tissue section, leave it for 30 min, then suck up the solution, stain with 100 μL and 15 μM of the above peptide probe solutions, and incubate at 4° C. for 2 h; After removing the staining solution, wash with 1×PBS for 3 times, and wash the unbound probe with the washing solution; Drop mount solution, add cover slide, observe and take photos with fluorescence microscope.

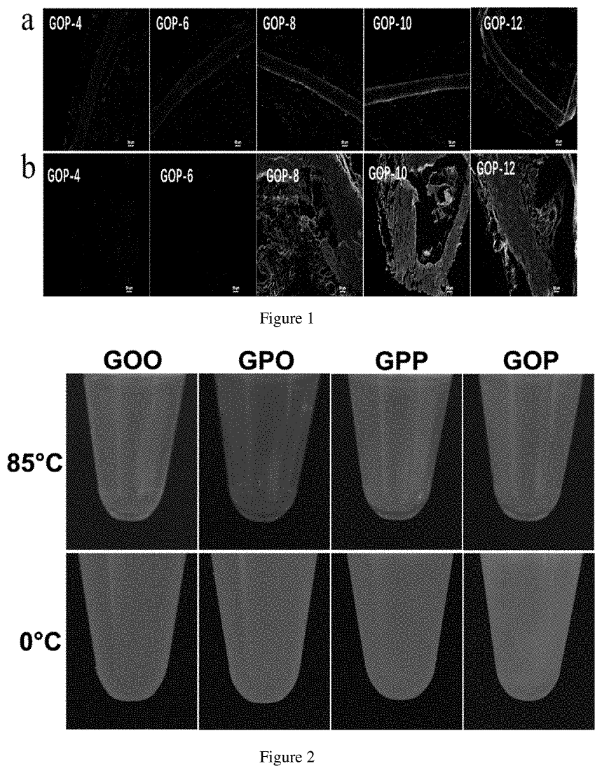

Results: The staining results of paraffin-embedded sternum tissues of peptide probe GOP-4, GOP-6, GOP-8, GOP-10 and GOP-12 prepared in Embodiment 1-5 of the present invention are shown in FIG. 1a. The staining results of frozen tail tissues are shown in FIG. 1b. The results show that the tissue stained by peptide probe with GOP repeated sequence less than 6 has no fluorescence, while the tissue stained by peptide probe with GOP repeat sequence greater than or equal to 8 can be detected with obvious fluorescence, which indicates that the peptide probe provided by the invention can be used to detect collagen.

2. Colorimetric Assay

Take the peptide probe GOP-10, GPP-10, GPO-10 and GOO-10, and prepare 300 μM probe solutions respectively; Keep above probe solutions in a water bath at 85° C. for 20 min, and collect picture data via a digital camera and observe the color of the solutions. Place heated probe solution in the ice-water mixture at 0° C. for 12 h, and then collect picture data via a digital camera again and observe the color of the solution. The results are shown in FIG. 2: Peptide probes GPP-10, GPO-10 and GOO-10 are trimer state (orange) at 0° C., and turn into single-stranded conformation (green) after heating; However, the peptide probe GOP-10 prepared by the invention maintains a stable single-stranded conformation (green) at 0° C., and is also in a single-stranded state (green) after heating.

3. Fluorescence spectrogram

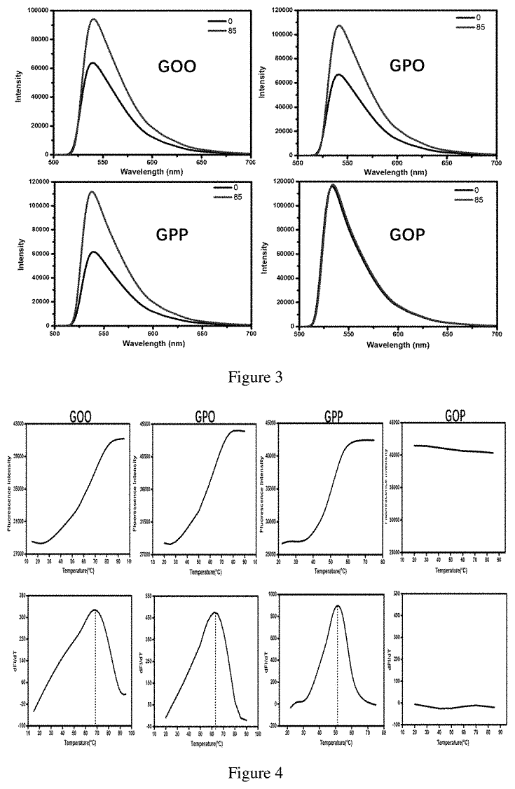

Take the peptide probe GOP-10, GPP-10, GPO-10 and GOO-10, and prepare 300 μM probe solutions respectively; Keep above probe solutions in a water bath at 85° C. for 20 min, and measure the fluorescence spectrogram by a fluorescence spectrophotometer immediately. Place heated probe solution in the ice-water mixture at 0° C. for 12 h, and measure the fluorescence spectrogram by fluorescence spectrophotometer again; The excitation wavelength is 497 nm, and the scanning range of emission spectrum is 500-700 nm. The results are shown in FIG. 3: After the peptide probes GPP-10, GPO-10 and GOO-10 were treated in 85° C. water bath and then placed in 0° C. ice bath, the fluorescence intensity changed significantly, indicating that these peptide probes changed from single-stranded state to trimer; However, after the peptide probe GOP-10 prepared by the invention is treated in an 85° C. water bath and then placed in an ice bath, the fluorescence intensity does not change, indicating that the peptide probe always maintains a single-stranded conformation.

4. Thermal stability test

Take the peptide probe GOP-10, GPP-10, GPO-10 and GOO-10, and prepare 300 μM probe solutions respectively; Dilute each probe solution to 15 μM by 10 mM phosphate buffer, and store at 0° C. for later use. Take the probe solutions respectively, raise the temperature from 0° C. to 95° C. in a gradient, and detect the fluorescence intensity of the solutions at 525 nm during the temperature rise process to obtain the fluorescence intensity-time curve; Conduct the first derivative of the curve by corresponding the maximum value of the curve to the thermal change temperature of the probe. The results are shown in FIG. 4: The fluorescence intensity of peptide probes GPP-10, GPO-10 and GOO-10 increased significantly in the range of 0-95° C., and the thermal transformation temperatures of GPP-10, GPO-10 and GOO-10 measured by derivative curves were 39° C., 63° C. and 69° C. respectively. The fluorescence intensity of the peptide probe GOP-10 prepared by the invention does not change in the range of 0-95° C., which indicates that the peptide probe GOP-10 prepared by the invention can keep a stable single-stranded state in the range of 0-95° C.

### Embodiment 7 Fluorescence Staining of Peptide Probe

1. Staining of collagen in intestinal tissue of mice by peptide probe

Take the peptide probe GOP-10, GPP-10, GPO-10 and GOO-10, and prepare 300 μM probe solutions respectively; Take the intestinal tissue samples of mice paraffin embedding and slice to 4 μm; After washing and deparaffinating the slices, treat the intestinal tissue with ultrapure water at 85° C. for 10 minutes to denature collagen. Add blocking solution to the tissue section, leave it for 30 min, then suck up the solution, stain with 100 μL and 15 μM of the above peptide probe solutions with different pre-treatment methods, and incubate at 4° C. for 4 h; After removing the staining solution, wash with 1×PBS for 3 times, and wash the unbound probe with the washing solution; Drop mount solution, add cover slide, observe and take photos with fluorescence microscope;

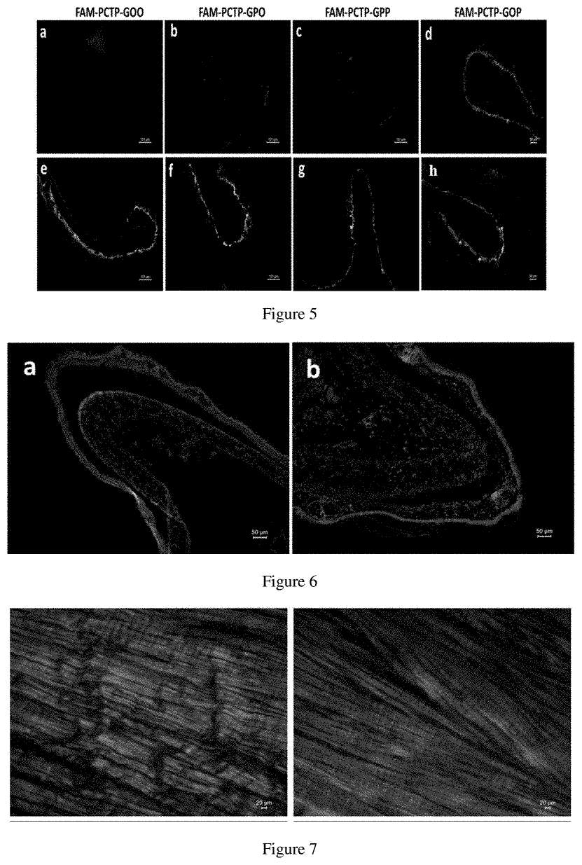

The results are shown in FIG. 5, in which the staining results of the peptide probe incubated at 0° C. before staining are shown in FIG. 5a-d; The staining results of the above peptide probes incubated at 85° C. before staining are shown in FIG. 5e-h; Peptide probes GPP-10, GPO-10 and GOO-10 can be fluorescently stained only after being pretreated at 85° C., but have no fluorescence staining ability at 0° C. The peptide probe GOP-10 prepared by the invention has good fluorescence staining ability after incubation at 85° C. and 0° C., which indicates that the peptide probe GOP-10 prepared by the invention still maintains a stable single-stranded state at 0° C., and it can be used for fluorescence imaging of collagen without heating pretreatment.

2. Staining of collagen in ear tissue of normal mice and diseased mice by peptide probe

Take the peptide probe GOP-10 and prepare 300 μM probe solutions;

Take mouse ear tissue samples for paraffin embedding and slice to 4 μm; After washing and deparaffinating the slice, treat part of the ear bone tissues with ultrapure water at 85° C. for 10 minutes to denature collagen, thus obtaining injured ear tissues; Untreated ear tissue is normal ear tissue. Add blocking solution to the tissue section, leave it for 30 min, then suck up the solution, stain with 100 μL and 15 μM of the above peptide probe solutions, and incubate at 4° C. for 4 h; After removing the staining solution, stain with 100 μL DAPI diluent for lmin and wash with 1×PBS for 3 times, and wash the unbound probe and excessive DAPI diluent with the washing solution; Drop mount solution, add cover slide, observe and take photos with fluorescence microscope;

The test results are shown in FIG. 6, in which the fluorescence detection results of injured mouse ear tissues are shown in FIG. 6a and normal mouse ear tissues are shown in FIG. 6b. In FIG. 6a, the pathological collagen in the injured mouse ear tissues is stained green, but there is no green fluorescence in FIG. 6b, only the nucleus stained by DAPI, which indicates that the peptide probe GOP-10 prepared by the invention only has targeting effect on the pathological collagen, and is not affected by the normal collagen at all, thus it can be used for the specific detection of the pathological collagen.

3. Staining of pathological section of human arthritis

Take human osteoarthritis tissue for paraffin embedding, slice to 4 μm, carry out washing and deparaffinating, and then add with 0.5 mL blocking solution, and suck off the liquid after placing for 30 min. Stain with 100 μL, 15 μM peptide probe solution, and incubate at 4° C. for 4 h. After removing the staining solution, stain with 100 μL DAPI diluent for 1 min. Use 1×PBS to wash for 3 times, and wash the unbound probes and excess DAPI. Drop mount solution, add cover slide, observe and take photos with fluorescence microscope.

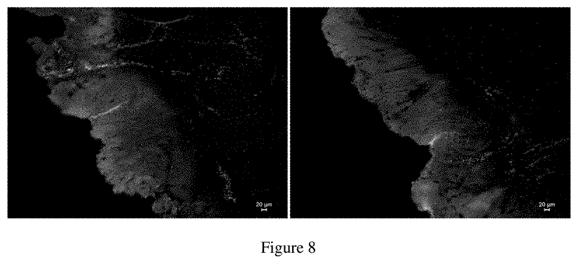

The staining results of pathological sections of fibrocartilage of human osteoarthritis are shown in FIG. 7, and the staining results of pathological sections of hyaline cartilage of human osteoarthritis are shown in FIG. 8. The probe GOP-10 can specifically bind to pathological collagen in osteoarthritis tissues, while it is stained green, and the nucleus is stained blue by DAPI. Therefore, the peptide probe GOP-10 prepared by the invention can be used for detecting pathological collagen in human osteoarthritis fibrocartilage and osteoarthritis hyaline cartilage.

1. (canceled)

2. (canceled)

3. (canceled)

4. (canceled)

5. (canceled)

6. (canceled)

7. (canceled)

8. (canceled)

9. (canceled)

10. (canceled)

11. A peptide probe for detecting pathological collagen is characterized in that it comprises a peptide sequence (Gly-Hyp-Pro)ₙ and a signal molecule X modified at the N-terminal of the peptide sequence (Gly-Hyp-Pro)ₙ, where n is an integer greater than 6.

12. The peptide probe as described in claim 11 is characterized in that the signal molecule X is fluorescein dye, coumarin dye, rhodamine dye, cyanine dye, BODIPY dye, tetraphenylethylene dye, and one or more of hexaphenylmethylsilane dyes, stilbene anthracene dyes, semiconductor quantum dots, carbon quantum dots, perovskite quantum dots, rare earth ion complexes, metal frame materials, up-conversion rare earth nanomaterials and long afterglow nanomaterials.

13. A peptide probe as described in claim 12 is characterized in that the signal molecule X is carboxyfluorescein FAM.

14. The peptide probe as described in claim 11 is characterized in that the signal molecule X and the peptide sequence (Gly-Hyp-Pro)ₙ are connected through a linker Ahx, and the peptide probe sequence is FAM-Ahx-(Gly-Hyp-Pro)ₙ.

15. The peptide probe as described in claim 14 is characterized in that the n is any integer between 8 and 12.

16. A method for preparing the peptide probe according to claim 11, which is characterized in that the methods include:
(1) Solid-phase synthesis of peptide resin (Gly-Hyp-Pro)ₙ;
(2) After 4eq of signal molecules, HOBt and HBTU are dissolved in DMF and activated at low temperature for 10-30 min, 4-10 eq of DIEA is added to the solution to obtain a mixture;
(3) Add the mixture prepared in step (2) to the peptide resin described from step (1) and react for 12-48 h away from light;
(4) After treating the peptide resin from step (3) with the cutting fluid for 2-4 h, add ice diethyl ether, and the resulting precipitate is referred to as the peptide probe. Wherein, the cutting fluid consists of trifluoroacetic acid, free radical catching agent and water in accordance with a volume ratio of 95:2.5:2.5.

17. Application of the peptide probe according to claim 11 in preparing a detection reagent, a kit and/or an imaging reagent for detecting pathological collagen.

18. A detection reagent containing the peptide probe of claim 11.

19. A detection kit containing the peptide probe of claim 11.

20. A tissue imaging reagent containing the peptide probe of claim 11.
